(19) United States
(12) Patent Application Publication
(10) Pub. No.: US 2017/0224048 A1
(43) Pub. Date: Aug. 10, 2017

(54) INJURY REDUCTION INSOLE

(71) Applicant: Victoria University, Footscray, VIC (AU)

(72) Inventors: Hanatsu Nagano, Southbank, VIC (AU); Rezaul Begg, Vermont, VIC (AU)

(21) Appl. No.: 15/328,956

(22) PCT Filed: Jul. 29, 2015
(86) PCT No.: PCT/AU2015/000451

§ 371 Date: Jan. 25, 2017

(30) Foreign Application Priority Data
Jul. 30, 2014 (AU) 2014902939

(51) Int. Cl. A43B 7/20; A43B 17/14; A61F 5/01; A43B 17/00; A61B 5/11; A43B 7/14; A43B 17/02

(52) U.S. Cl. CPC A43B 7/20; A43B 7/141; A43B 17/14; A43B 17/02; A43B 17/006; A43B 7/146; A43B 7/1445; A61B 5/112; A61F 5/0111

(57) ABSTRACT

A footwear-insole for modifying a user's gait configured to extend under a user's foot and provide greater than 0 to 5.0° dorsiflexion support and greater than 0 to 5.0° eversion support.

### PRIORITY CROSS-REFERENCE

This application claims priority from Australian Provisional Application No. 2014902939 filed on 30 Jul. 2014, the contents of which are to be taken as incorporated herein by this reference.

### TECHNICAL FIELD

The present invention generally relates to a footwear insole for modifying a user's gait. The modified gait can reduce injuries that may occur during locomotion, such as walking. The invention is particularly applicable for reducing injuries associated with falls, acute ankle sprain and knee osteoarthritis (which may develop as a result of locomotion-related long-term wear and tear on joints) and it will be convenient to hereinafter disclose the invention in relation to that exemplary application. However, it is to be appreciated that the invention is not limited to that application and could be used in other foot orthotic applications.

### BACKGROUND OF THE INVENTION

The following discussion of the background to the invention is intended to facilitate an understanding of the invention. However, it should be appreciated that the discussion is not an acknowledgement or admission that any of the material referred to was published, known or part of the common general knowledge as at the priority date of the application.

Mobility is important to keep older adults (aged over 65 years old) healthy. Walking outdoors or on a walking machine such as treadmill is a recommended exercise to maintain an active lifestyle. However, walking can lead to falls, acute and long-term overuse injuries, and lower limb degeneration (for example ulcers, foot deformities and joint pain). It is therefore important for older adults to walk both actively and safely.

Falls among older adults are recognised as a significant healthcare issue worldwide. One in every three older adults falls every year, and 9 to 20% of those incidences result in severe injuries, such as hip fractures. Falls have a negative impact on an individual's quality of life. For example, half of the hip fractures due to falling result in permanent loss of an independent lifestyle and 20 to 30% of falls eventually result in death.

Falls-related injuries are estimated to cost approximately AU$3 billion per year in Australia, and the average cost per falls-related hospitalisation of an individual in Australia is reported to be over AU$20,000. Medical costs are predicted to triple in the next 40 years due to the increasing proportion of older adults relative to the population as a whole, from 13.5% of the population in 2010 to 22% by 2050.

Establishment of an effective and innovative intervention has the potential to impact positively on the falls problem worldwide. In Australia, every 1% reduction in the rate of falls could prevent 45,000 older adults (over 65 years old) from falling and could save associated medical costs of AU$90 million every year. Equivalent medical cost saving figures are reported to be AU$900 million and AU$245 million in the U.S.A. and Japan, respectively.

Various intervention strategies have been proposed to reduce the risk of falls (e.g. strength training). However, without constant supervision and encouragement (e.g., personal trainers, rehabilitation centres) older adults are unlikely to participate in activities which reduce the risk of falls unless all the following requirements are met: low cost, effortless, immediate effect and easy application.

It is therefore desirable to provide an aid or device which is capable of assisting with one or more of the above identified problems.

### SUMMARY OF THE INVENTION

A first aspect of the present invention provides a footwear-insole for modifying a user's gait. The footwear insole is configured to extend under a user's foot and provide greater than 0 to 5.0° dorsiflexion support and greater than 0 to 5.0° eversion support

The present invention provides biomechanical technology for footwear-insoles (“insoles”) that modifies the gait of a person. The insole of the present invention provides a specific incline on the footwear-insole interface to modify ankle joint orientation inside the footwear (for example a shoe). Gait can therefore be controlled in a way that can reduce the risk of injuries caused by tripping, falling (for example through loss of balance) and lower limb injuries during locomotion, such as walking. The present invention may be categorised as an “orthotic”, a specifically designed footwear-insole(s).

It should also be understood that dorsiflexion is that motion that brings the foot towards the lower leg (shank). Dorsiflexion is the primary ankle joint motion that is used to maintain sufficient swing foot-ground clearance in order to reduce the risk of tripping. Furthermore, it should also be understood that eversion is that motion denoting the movement of the sole of the foot away from the midline of the body so sole of the foot faces outward. Eversion can improve sideways balance by regulating lateral movement of the centre of pressure (CoP) under the foot and the body centre of mass (CoM).

The insole is designed to assist with ankle joint dorsiflexion during locomotion and to provide eversion support for tripping risk minimisation and improved sideways balance. Therefore, the dorsiflexion support angle is preferably selected to assist with ankle joint dorsiflexion during locomotion. In some embodiments, the insole provides from 0.5° to 3.5° dorsiflexion support, preferably from 1.0° to 3.0° dorsiflexion support, more preferably from 2.0° to 2.5° dorsiflexion support, and yet more preferably about 2.2° dorsiflexion support. Furthermore, the eversion support angle is preferably selected to provide improved sideways balance during locomotion. In some embodiments, the insole provides from 2.0° to 5.0° eversion support, preferably from 3.0° to 5.0°, more preferably from 3.5° to 5.0° eversion support eversion support, yet more preferably from 4.0° to 5.0° eversion support, and yet more preferably about 4.5° eversion support. It is noted that in previous research, older adults have demonstrated reduced (5.3°) dorsiflexion range of motion compared to young adults during walking.

It should be understood that locomotion of a user encompasses a number of types of motions including walking, jogging, running, jumping and the like. In the preferred embodiment, the footwear-insole for reducing injuries that may occur from or during walking.

The insole can also improve mechanical energy efficiency, which may be useful for relieving conditions such as lower limb joint osteoarthritis. Additionally, locomotion such as walking is a repetitive movement, and as a result any suboptimal gait characteristics should be modified to avoid eventual onset of long-term overuse injuries. The use of a footwear-insole can therefore correct subtle gait problems which, without modification, can lead to significant problems over time.

A common long-term overuse injury is osteoarthritis of the lower limb joints, especially at the knees. One major cause of knee osteoarthritis is poor oscillation of impact which is generated at the initial foot contact. The efficient loading maximises the oscillation of this impact to initiate the opposite limb's toe-off. Without sufficient reliance on this mechanism, impact is transferred as a shock-wave to the articular cartilages of the knee joint and can potentially progress to osteoarthritis. Dorsiflexion and eversion ankle joint motions at heel contact can assist energy efficient loading and thus help to minimise gradual damage to the knee joint.

The dorsiflexion support and eversion support of the insole can extend over part of, or all of the insole. In some embodiments, the amount and thus angle of dorsiflexion support and eversion support varies across the insole. However, in preferred embodiments, the amount of dorsiflexion support and eversion support is substantially constant along and across the surface of the insole. For example, in some embodiments the insole extends from the heel to the outer toe of a user's foot. In these embodiments, the insole preferably provides a longitudinal and a lateral inclination from an inner heel section of the insole to an outer toe section of the insole to respectively provide dorsiflexion and eversion support.

The inclination of the insole is typically measured relative to a base of the insole. The base of the insole is configured to seat on top of an inner base portion of footwear in which the insole is inserted. In preferred embodiments, the insole includes a substantially planar base and a top surface, the longitudinal and the lateral inclination being formed between the base and the top surface of the insole. The base therefore provides a flat bottom surface for the insole and the top surface provides an inclined surface on which a user's foot rests and is then angled to provide the requisite dorsiflexion support and eversion support. In such embodiments, it is preferred the inclination of the top surface providing the dorsiflexion support and eversion support is substantially constant across the top surface of the insole. In some embodiments, the longitudinal inclination (dorsiflexion angle) and the eversion angle lateral inclination (eversion angle) is substantially the same across the entire insole where surface contours are not a substantial factor. In this respect, dorsiflexion and eversion angles on the insole surface may vary slightly to some part of the insole due to such factors as individual-specific foot moulding, arch support, comfort, foot shapes, conditions or the like.

In some embodiments, the insole includes the following reference points:
A=the most inner medial part of the insole within one fifth of the posterior part of the insole;
B=the most inner medial part of the insole within one-fifth of the anterior part of the insole;
C=the most outer lateral part of the insole within one-fifth of the posterior part of the insole; and
D=the most outer lateral part of the insole within one-fifth of the anterior part of the insole.
   In these embodiments, it is preferred for the dorsiflexion angle of A to B is approximately equal to the dorsiflexion angle of C to D; and the eversion angle of A to C is approximately equal to the eversion angle of B to D.

The present invention is typically inserted into footwear as an insole therein. The insole is therefore preferably configured to be generally foot shaped in order to fit within that footwear. It should be understood that by generally foot shaped element it is meant that the insole takes the general shape of a person's foot, similar to the shape of a shoe. Like a shoe, not all of the detail of the foot is necessary, for example the individual shape of the toes, to provide this general foot shape. A generally foot shaped element can have the general outline of the foot, with a general arch or other shape for the toe region of the foot. The element should be suitably shaped to cover the bottom surface of a person's foot.

The insole is preferably configured to seat under a user's foot. This can be achieved in a number of ways. In preferred embodiments, the insole is configured to be inserted into footwear. Suitable footwear includes a shoe, boot, and the like. Preferably, the insole is shaped to fit a particular footwear shape and configuration, for example within a particular shoe or boot. It should be appreciated that the sizing of the insole may vary according to the shape and length of the subject's foot. The insole may therefore be personally shaped to the user's unique foot anatomy, any pathology and to the geometry of any specific shoe. It should be appreciated that in some embodiments, the insole may be made-to-measure or tailored for an individual, whilst in other embodiments the insole may be a manufactured item where adaptation to the individual is only the choice of size and/or choice of insole angling.

Patients or users of the insole would typically use an insole in each of the left and right items in a pair of footwear. The present invention therefore can comprise a pair of insoles, a left foot insole and a right foot insole, wherein the dorsiflexion support and eversion support are tailored for each foot of a user. For example, if tripping risk is diagnosed greater for one foot than the other, more dorsiflexion support may be applied to assist the affected foot. For one knee suffering from severe osteoarthritis, appropriate dorsiflexion and eversion can be prescribed for that limb to enhance mechanical energy efficiency for impact relief on the knee. Additionally, ankle support can be varied to provide different degrees of ankle joint angles support into each insole to correct any asymmetry in gait control or lower limb joints' conditions.

The insole of the present invention can be formed from any suitable material. In some embodiments, wherein the insole includes at least one layer of a viscoelastic material, preferably a closed cell foam, more preferably an ethylene vinyl acetate (EVA) foam. Viscoelastic materials exhibit both viscous and elastic characteristics when undergoing compression. Viscous materials resist strain linearly with time when a stress is applied. Elastic materials strain instantaneously when compressed, and quickly return to their original state as the stress is removed. Viscoelastic materials possess elements of both of these properties and exhibit time dependent strain. These materials may be obtained from suppliers known in the foam and plastic arts. EVA is a polymer found to provide desirable elastomeric properties and provides desired softness and flexibility.

In some embodiments, the insole can comprise a single layer of viscoelastic material. In other embodiments, the insole can comprise a multilayer construction having one or more layers of viscoelastic material. In some embodiments, the multilayer construction may also include layers of other materials, such as other polymers (including plastics and/or foams), gels or the like.

In embodiments that include plastic materials, the plastic materials can be, for example, thermosets, such as, for example, alkyd polyesters, allyls, bakelite, epoxy, melamine, phenolics, polybutadienes, polyester, polyurethane, silicones, ureas, and the like. Likewise, the plastic materials can include bioplastics. Bioplastics are a form of plastics derived from renewable biomass sources, such as vegetable oil, corn starch, pea starch, or microbiota, rather than traditional plastics that are often derived from petroleum. Types of bioplastics suitable for use with embodiments of the invention include, for example, polylactide acid (PLA) plastics, poly-3-hydroxybutyrate (PHB), polyimide 11 (PA 11), bio-derived polyethylene, and the like. Such materials are known in the plastic arts and can be moulded according to known methods such as injection moulding and the like.

In embodiments that include foam materials, the foam can be, for example, polyurethane foam (foam rubber), polystyrene foam, or the like. In embodiments utilizing polyurethane foam, the type of polyurethane foam can be, for example, elastomers, including, EPM (ethylene propylene rubber, a copolymer of ethylene and propylene) and EPDM rubber (ethylene propylene diene rubber, a terpolymer of ethylene, propylene and a diene-component), Epichlorohydrin rubber (ECO), Polyacrylic rubber (ACM, ABR), Silicone rubber (SI, Q, VMQ), Fluorosilicone Rubber (FVMQ), Fluoroelastomers (FKM, and FEPM) Viton, Tecnoflon, Fluorel, Aflas and Dai-El, Perfluoroelastomers (FFKM) Tecnoflon PFR, Kalrez, Chemraz, Perlast, Polyether Block Amides (PEBA), and Chlorosulfonated Polyethylene (CSM). Depending on the characteristics of the foam, it may be acceptable to combine a soft foam or open cell foam over hard or rigid foam to produce a cushioned wedge. In embodiments utilizing polystyrene foam, the type of polystyrene foam can be, for example, expanded polystyrene foam, and extruded polystyrene foam, or the like. In embodiments of extruded polystyrene foam (XPS), the XPS foam can be, for example, Styrofoam, or the like.

In some embodiments, the insole can comprise a multi-material construction having sections of the insole constructed from different materials. For example, in one embodiment that insole comprises a first material in the heel section to mid-foot, and a second material in the front half (for example from mid-foot to the toes) of a lighter material. This construction could potentially reduce tripping risks. Similarly, in some embodiments the section of the insole which seats or is proximate to the toes of a user can be formed from a material with the better resilience and therefore potentially improve energy efficiency, possibly preventing fatigue onset and knee pain of a user.

Excessive foot pressure on the metatarsal region and the big toe is known to cause foot deformities and ulcers. The application of foot-moulding technology into these insoles can further assist with the re-distribution of excessive foot plantar pressures, which in turn may help to prevent foot ulcer formation. Therefore, in some embodiments at least a portion of the top surface of the insole is moulded to fit a user's foot shape. Foot-moulding technology can accommodate individual foot shapes such as arch-support or the like. Thus, in some embodiments, the top surface of the insole includes arch support, preferably moulded arch support.

Moulding technology, or more cost-effective semi-moulding technology can maximise the foot contact area with the interface (e.g. arch-support), and thus foot pressure can be more widely distributed. Dorsiflexion motion in general is also an effective ankle motion for shifting foot pressure to a more posterior position, thus relieving a common site of ulcers (i.e. the big toe and the metatarsal region).

In some embodiments, the insole interface can be textured to enhance afferent feedback for quicker reaction. Use of a textured surface on the footwear-insoles can stimulate cutaneous receptors on the bottom of the foot and may improve movement control. The use of these textures on footwear-insoles improves reaction speed and assists balance recovery whilst walking. Therefore, in some embodiments the insole includes a top surface which includes at least one textured section. The textured surface is configured to stimulate cutaneous receptors to promote afferent feedback for quicker reaction.

The textured surface can have any suitable configuration, including length and size. The selection of texture, height and diameter/area of the insole applied depend on an individual's foot condition and the level of sensation to be produced. The number of textures used, spacing between multiple textures and diameter or material of a texture determine the level of tactile sensation required for different individuals. In some embodiments, the textured section has a texture height of from 0.5 to 20 mm preferably from 1 mm to 20 mm. In some embodiments, the textured section has a diameter from 2 mm to 30 mm, preferably from 5 mm to 30 mm. In some embodiments, the textured sections are configured on the top surface of the insole in at least one of the following configurations:

at least two, preferably multiple, textured sections aligned along the center of pressure (CoP) path of the insole;

at least two parallel lines of multiple textured sections aligned along and with the CoP path of the insole;

at least two textured sections located in a Metatarsal region of the insole. The addition of textured surfaces to the Metatarsal region accentuates tactile sensation at metatarsal region in case dorsiflexion support reduces sufficient afferent feedback;

an array of textured sections laterally spread along one lateral half of the insole. The addition of laterally displaced textured surfaces can assist response for lateral balance loss by activating a greater cutaneous sensation when CoP is dislocated more laterally. A tactile sensation is activated when CoP is laterally deviated designed particularly for prevention of sideway balance disturbance.

It should be appreciated that the configuration of the texture sections is different on the insoles of a user's two feet. This enables the textured surface to be tailored for a user's needs and the individual tactile sensitivity of each foot.

In some embodiments, modification of the heel section of the insole could potentially prevent the foot inside the shoe from sliding on the insole's inclined surface. For example, the heel section of the insole may include a recess formation or other depression which seats a user's heel (of their foot) therein, reducing the tendency of the foot sliding forward in a shoe.

The present invention can function to minimise the risk of falls by minimising tripping and loss of sideways balance by improving dorsiflexion and providing eversion ankle joint motion support. Thus, a second aspect of the present invention provides a footwear-insole for reducing the risk of falling from or during locomotion from at least one of tripping or loss of balance, preferably loss of sideways balance, the footwear insole is configured to extend under a user's foot and provide greater than 0 to 5.0° dorsiflexion support and greater than 0 to 5.0° eversion support.

A third aspect of the present invention provides a method of controlling the gait of a patient to reduce the risk of a patient falling from or during locomotion comprising providing at least one footwear-insole according to the first aspect of the present invention or the second aspect of the present invention in a user's shoe. Locomotion injuries from falling can be a result of a number of incidences including at least one of tripping, or loss of balance, for example loss of sideway balance. Furthermore, the insole can improve mechanical energy efficiency, which may be useful for relieving conditions such as lower limb joint osteoarthritis.

A fourth aspect of the present invention provides a method of modifying the gait of a patient to reduce the risk of falling from or during locomotion comprising:
measuring movement of a patient's minimum foot clearance (MFC) to determine a suitable dorsiflexion support angle to enhance ankle joint dorsiflexion and thereby increase swing foot clearance of the patient at MFC during locomotion;
measuring lateral movement of a patient's body centre of mass (CoM) during locomotion to determine an eversion support angle which redirects the CoM away from a lateral safety boundary toward the opposite foot; and
providing a footwear insole in a user's footwear configured to extend under a user's foot which provides the determined dorsiflexion support angle and eversion support angle, wherein the footwear insole provides greater than 0 to 5.0° dorsiflexion support and greater than 0 to 5.0° eversion support.

A fifth aspect of the present invention provides a method of modifying the gait of a patient to reduce the risk of falling from or during locomotion comprising:
measuring a patient's gait to determine movement of the patient's minimum foot clearance (MFC) and the patient's lateral movement of the body centre of mass (CoM) during locomotion;
using the patient's minimum foot clearance (MFC) characteristics to determine a suitable dorsiflexion support angle to enhance ankle joint dorsiflexion and thereby increase swing foot clearance of the patient at MFC;
using the patient's lateral movement of the centre of mass (CoM) to determine an eversion support angle which redirects the CoM away from a lateral safety boundary toward the opposite foot; and
providing a footwear insole in a user's footwear configured to extend under a user's foot which provides the determined dorsiflexion support angle and eversion support angle, wherein the footwear insole provides greater than 0 to 5.0° dorsiflexion support and greater than 0 to 5.0° eversion support.

The method of this fourth and fifth aspect of the present invention therefore enables customisation of a footwear-insole to a patient's specific requirements. As noted above, increasing a patient/user's swing foot clearance at MFC reduces tripping risk at MFC and redirecting the CoM away from a lateral safety boundary toward the opposite foot increases the patient's sideways balance. Combined, these two improvements reduce the fall risk for that patient and increases safety during locomotion, for example walking.

It should be appreciated that the insole of the methods of the fourth and fifth aspect of the present invention can have any of the features defined for the footwear insole of the first aspect of the present invention.

The present invention is useful for solving various problems associated with locomotion such as walking including minimising the risk of falls, preventing ankle sprains and reducing impact on lower limb joints. Although older adults would likely be the main beneficiaries of the invention, it could be expanded to other population groups with gait problems such as those who are obese, or who suffer from cerebral palsy, Parkinson's disease or who have suffered a stroke.

### BRIEF DESCRIPTION OF THE DRAWINGS

The present invention will now be described with reference to the figures of the accompanying drawings, which illustrate particular preferred embodiments of the present invention, wherein:

FIG. 1 provides a schematic of a right foot insole according to one embodiment of the present invention showing the inclination of the insole surface of the present invention and points of support dorsiflexion and eversion.

FIG. 2 provides an example of insole moulding according to one embodiment of the present invention, in which (A) shows an example of moulding to account for an individual's arch-support; and (B) shows an example of the moulded insole surface.

FIG. 3 provides four examples (A) to (D) of variations of texture installation on an insole according to the present invention which provide enhanced reaction speed.

FIG. 4 illustrates how dorsiflexion support reduces tripping risks at minimum foot clearance (MFC), showing (A) a schematic of MFC event illustrating the local minimum of vertical swing foot displacement at mid-swing; (B) a plot of the typical swing foot clearance vs swing time; and (C) an illustration of how dorsiflexion support could increase swing foot clearance to avoid obstacle contact.

FIG. 5 provides an illustration of sideway balance and minimum lateral margin (MLM).

FIG. 6 provides an illustration of centre of pressure (CoP) control and prevention of inversion ankle sprain by the eversion support, showing (A) a photograph showing an example of inversion ankle sprain; (B) a photograph showing an example of eversion ankle motion; and (C) a schematic illustration of lateral CoP displacement.

FIG. 7 is an illustration of an embodiment of the insole according to one embodiment of the present invention used for experimental test runs. The insole was 26 cm in length and offers 2.2° dorsiflexion and 4.5° eversion support.

FIG. 8 illustrates the insole effects on ankle angle (dorsiflexion/plantarflexion) at MFC for young and older adults, dominant and non-dominant limbs separately.

FIG. 9 illustrates the insole effects on Minimum Foot Clearance (MFC) for young and older adults, dominant and non-dominant limbs separately.

FIG. 10 illustrates the insole effects on Minimum Lateral Margin (MLM) and lateral Centre of Pressure (CoP) displacement for young and older adults, dominant and non-dominant limbs separately.

FIG. 11 illustrates the insole effects on recovery rate, time to foot flat and peak knee adduction moment for young and older adults, dominant and non-dominant limbs separately.

FIG. 12 illustrates the insole effects on foot contact angle at heel contact for young and older adults, dominant and non-dominant limbs separately. Foot contact angle at heel contact is formed by the toe, heel and the floor surface.

FIG. 13 illustrates the insole effects on basic spatio-temporal gait parameters in young and older adults (effects on dominant and non-dominant limbs have been shown separately).

FIG. 14 illustrates the insole effects on shank-floor contact angle at heel contact for young and older adults, dominant and non-dominant limbs shown separately.

### DEFINITION OF TERMS USED

Falls in the context here are defined as “inadvertently coming to rest on the ground, floor or other lower level, excluding intentional change in position to rest in furniture, wall or other objects”.

Tripping is defined as an event in which the most distal feature of the swing limb, usually the lowest part of the shoe or foot, makes unanticipated contact with either the supporting surface or objects on it with sufficient force to destabilise the walker.

Sideway balance loss is defined when the Centre of mass (CoM) is dislocated lateral to the stance foot's boundary.

Minimum foot clearance (MFC) is defined as the local minimum of vertical swing foot-ground clearance during mid-swing phase of the gait cycle (FIG. 1).

Minimum Lateral Margin (MLM) is the event when medio-lateral distance between the CoM excursion and the stance foot is minimum (FIG. 5).

Centre of Mass (CoM) is the body's centre point in three dimensions determined from the individual body segments' CoM.

Centre of Pressure (CoP) is the net location of the resultant foot-ground reaction forces on the horizontal plane.

Stance foot is the foot in contact with the walking surface.

Swing foot is the foot off the walking surface and travelling forward during normal gait cycle.

### DETAILED DESCRIPTION

The present invention provides a footwear-insole for reducing injuries that may occur when walking, the footwear-insole is configured to extend under a user's foot and provide a range of dorsiflexion (greater than 0 to 5.0°) and eversion support (greater than 0 to 5.0°). The ankle joint supporting functions of dorsiflexion and eversion support for improved gait due to reduced risk of falls and greater mechanical energy efficiency have not been previously applied or tested into footwear-insoles. An insole having both dorsiflexion and eversion support according to the present invention is the first to examine the effects of inclination of the insole interface on optimum gait control.

A footwear-insole 100, in this case a right foot's insole according to one embodiment of the present invention, is illustrated in FIG. 1. The insole 100 comprises a generally foot shaped element having a planar base 102 and a top surface 104. The top surface 104 is inclined relative to the base 102 to provide the required dorsiflexion support and eversion support. The base 102 provides a flat bottom surface for the insole 100 and the top surface 104 provides an inclined surface on which a user's foot rests and is then angled to provide the requisite dorsiflexion support and eversion support. The insole 100 of the present invention therefore provides a specific incline on the footwear-insole interface to modify ankle joint orientation inside the footwear (for example a shoe). Gait can therefore be controlled in a way that can reduce the risk of tripping and falling.

Whilst not essential, it is preferred for the inclination of the top surface 104 relative to the base 102 providing the dorsiflexion support and eversion support is substantially constant across the top surface 104 of the insole 100. However it should be appreciated that the dorsiflexion and the eversion angles on the insole 100 surface may vary to slightly to some part of the insole 100 due to such factors as individual-specific foot moulding, arch support, comfort, foot shapes, conditions or the like.

As shown in FIG. 1, the top surface 104 provides inclination from the inner heel part toward the outer toe to support dorsiflexion and eversion of up to 5 degrees, depending on a wearer's foot condition, comfort and preference. The baseline height at the inner heel varies from 0.1 cm to 3 cm.

This inclination can be measured relative to specific locations on the insole 100. In the insole 100 shown in FIG. 1 the marked reference locations are:
A=the most inner medial part of the insole 100 within one fifth of the posterior part of the insole 100,
B=the most inner medial part of the insole 100 within one-fifth of the anterior part of the insole 100,
C=the most outer lateral part of the insole within one-fifth of the posterior part of the insole 100,
D=the most outer lateral part of the insole 100 within one-fifth of the anterior part of the insole 100,
P=the most posterior part of the insole 100 top surface 104,
F=the most frontal (anterior) part of the insole 100 top surface 104.

Both dorsiflexion (α) and eversion (β) angles are in the range, greater than 0° and less than 5°. Dorsiflexion angle (α) of A-B is approximately equal to the angle of C-D; eversion angle (β) of A-C is approximately equal to the angle of B-D. While the same inclination is applied to the entire top surface 104 relative to the base 102, dorsiflexion and eversion angles on the top surface 104 may vary to slightly to some part of the insole 100 due to such factors as individual-specific foot moulding, comfort, foot shapes and conditions.

The insole 100 may be configured for placement in footwear such as a shoe, boot, or the like as needed (not illustrated). It should be appreciated that the sizing of the insole 100 may vary according to the shape and length of the subject's foot. Furthermore, the appropriate thickness of insole 100 may involve considerations of the footwear type and configuration, activity application (walking, running, sports or the like), patient's age, weight, condition of the knee, ankle, hip and the like. Whilst this should not be considered as being limiting to the present invention, it is noted that in some cases insoles 100 having greater thickness may be desired when participating in sporting activities resulting in higher loads on the body.

The material of the footwear-insole 100 of the present invention can influence properties of the insole 100 such as elasticity, density, or resilience to maintain the inclination and functions. The insole 100 is therefore preferably formed of a viscoelastic material, preferably a viscoelastic foam. The viscoelastic material can be made at least in part from of any suitable cushioning material with the described properties and characteristics. That is, while the material provides a cushioning it also must retain a wedged shape, even when compressed. Preferably, the material has sufficient durometer (hardness) and possesses a physical memory, meaning that it returns to its original shape after the forces of compression are removed, readying it to accept the impact of the patient's next step and provide cushioning. The viscoelastic material enables the insole 100 to partially collapse under compressive forces and rebound when the compressive forces are removed.

An example of one suitable viscoelastic material is ethylene vinyl acetate (EVA) foam with its density varying depending on its location under the foot. In preferred embodiments the viscoelastic material is EVA foam or modification thereof. EVA foam provides a plurality of encapsulated gas pockets in the form of closed cells, which when surrounded by the EVA can mimic fatty globules surrounded by fibrous tissue found in the foot. As such, it has been found that EVA foam can be used to mimic the natural anatomical protective structures of the foot. There is a soft thin material covering the surface for comfort and security.

Moulding technology, or more cost-effective semi-moulding technology can maximise the foot contact area with the interface (e.g. arch-support), and thus foot pressure can be more widely distributed on the insole 100. Various custom-moulding methods can be applied to the top surface of the insole 100 to produce customised fit and support (for example arch support) to the feet of each individual user. This produces an individually moulded insole surface which takes into account each individual's foot shapes including arch-support.

It should be appreciated that the effects of custom-moulding technology for optimum foot pressure distribution is widely accepted in the art, while dorsiflexion support by orthotics has been also proven to offload the concentration of plantar pressure (for example, Bus, S. A., Ulbrecht, J. S., & Cavanagh, P. R. (2004). Pressure relief and load redistribution by custom-made insoles in diabetic patients with neuropathy and foot deformity. Clinical Biomechanics, 19: 629-638).

A large number of custom moulding methods are known in the art, including plaster casting or other casting technique, 3D foot scanning, self-moulding methods or the like. It should be appreciated that any number of these well known methods could be used in conjunction with the present invention.

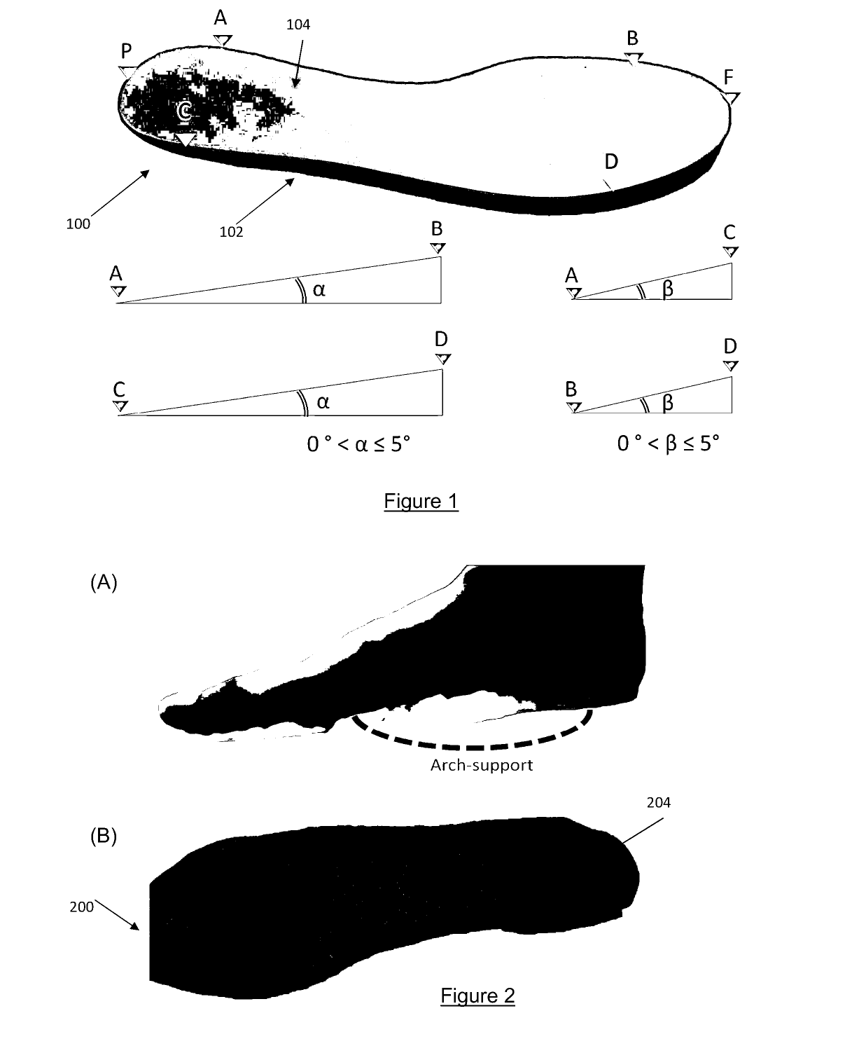

For example, FIG. 2 illustrates an example of a simple self-moulding method which takes into account an individual's arch-support. Self-moulding materials comprise a shape conforming material such as a thermo-moulding foam or another mouldable material the like, which moulds to a foot shape when pressure and/or heat is applied to an insole 200, for example as shown in FIG. 2(A) which shows the moulding technique. The resulting top surface 204 forms with matching contours to the foot applied to that insole 200, for example as shown in FIG. 2(B) which shows the resulting moulded insole top surface 204.

Other semi-custom moulding technology can alternatively be used based on a combination of heat-moulding and cryogenic freezing for polymer foams. Moulding can be performed when the material is at a lower density and higher temperature followed by sudden freezing, for example, using liquid nitrogen to fixate the moulded shape semi-permanently, for example as taught in Crabtree, P., Dhokia, V. G., Newman, S. T., & Ansell, M. P. (2009). Manufacturing methodology for personalised symptom-specific sports insoles. Robotics and Computer-Integrated Manufacturing, 25: 972-979.

As previously noted, the top surface of the insole 100 (foot to insole interface) can be textured to provide a greater tactile sensation and thus enhance reaction speed/feedback to user. Use of a textured surface on the footwear-insoles 100 can stimulate cutaneous receptors on the bottom of the foot and may improve movement control. It should be appreciated that texture installation on the CoP path has been previously reported to effectively enhance afferent feedback by providing greater tactile sensation for cutaneous receptors that are aligned on the typical CoP path (for example Nurse, M. A., & Nigg, B. M. (2001). The effect of changes in foot sensation on plantar pressure and muscle activity. Clinical Biomechanics, 16: 719-727).

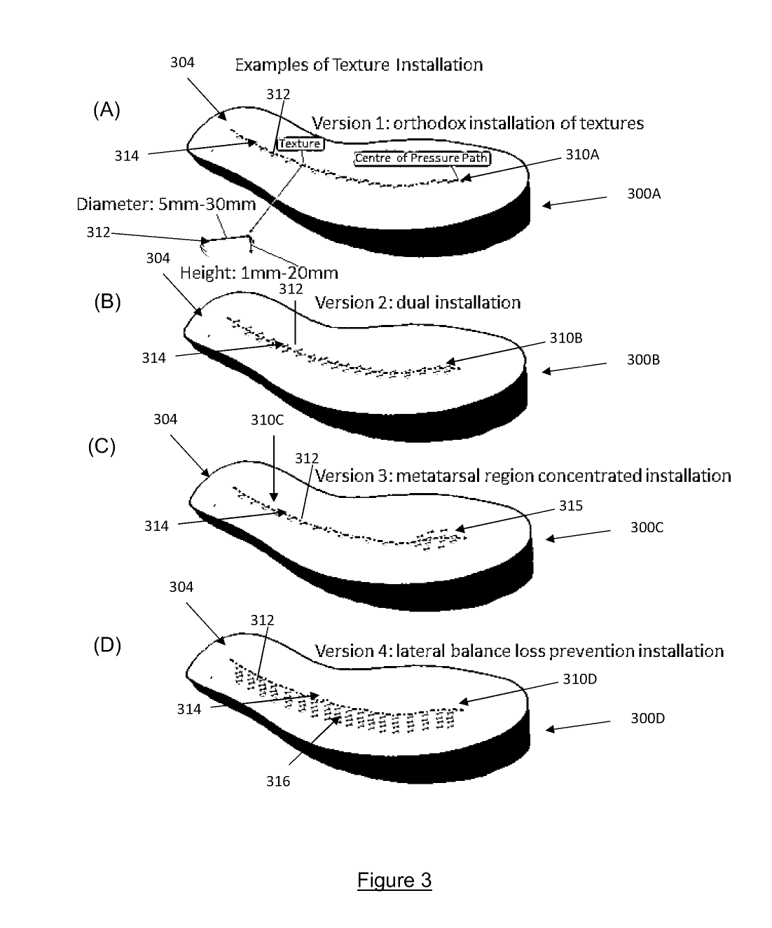

FIG. 3 provides four examples of texture installation 310A, 310B, 310C and 310D applied to an insole 300A, 300B, 300C 300D of the present invention for enhanced reaction speed. As shown in FIG. 3A, each texture installation 310A, 310B, 310C and 310D comprises multiply arranged texture elements 312 which comprise a circular area of textured material. Each texture element 312 has a texture specification based on height (1 mm to 20 mm) and diameter (5 mm to 30 mm). As should be appreciated, the exact specification would be selected to suit an individual's foot condition and the level of sensation to be produced. Similarly, the number of texture elements 312 used, spacing between multiple texture elements 312 and diameter or material of a texture element 312 determines the level of tactile sensation required for different individuals. As is understood in the art, various plastic, rubber and/or wooden materials could be used to enhance or maintain the functionality of a texture element 312. The fundamental concept is to stimulate cutaneous receptors to promote afferent feedback for quicker reaction. The illustrated texture installation 310A, 310B, 310C and 310D in FIG. 3 are as follows:

FIG. 3A shows texture installation 310A (Version 1) comprising an orthodox type with multiple texture elements 312 installed or mounted along with the CoP path 314.

FIG. 3B shows texture installation 310B (Version 2) comprising a dual installation (two parallel spaced apart lines of multiply aligned texture elements 312) extending along with the CoP path 314. The dual installation texture elements 312 strengthen the response sensation to a user.

FIG. 3C shows texture installation 310C (Version 3) comprising multiple texture elements 312 installed or mounted along with the CoP path 314 with a concentration of texture elements 312 in the Metatarsal region 315 of the insole. This texture installation 310C accentuates tactile sensation at metatarsal region 315 in case dorsiflexion support reduces sufficient afferent feedback.

FIG. 3D shows texture installation 310D (Version 4) comprising multiple texture elements 312 installed or mounted in an array 316 on one lateral side of the CoP path 314. This texture installation 310D is advantageous for lateral balance loss prevention to activate greater cutaneous sensation when CoP is dislocated more laterally. Version 4 aims to activate tactile sensation when CoP is laterally deviated designed particularly for prevention of sideway balance disturbance.

It should be appreciated that the texture installation on an insole 100, 200, 300A to 300D may differ between the two feet depending on tactile sensitivity. Thus, the present invention can be used to overcome any unwanted asymmetry by adopting different modifications on each side of footwear-insole 100, 200, 300A to 300D.

Examples of some of the various applications and functions of the insole of the present invention will now be discussed:

### Prevention of Tripping Risk at Minimum Foot Clearance (MFC) by Dorsiflexion Support

The insole of the present invention can reduce the occurrence of tripping, particularly tripping risks at minimum foot clearance (MFC).

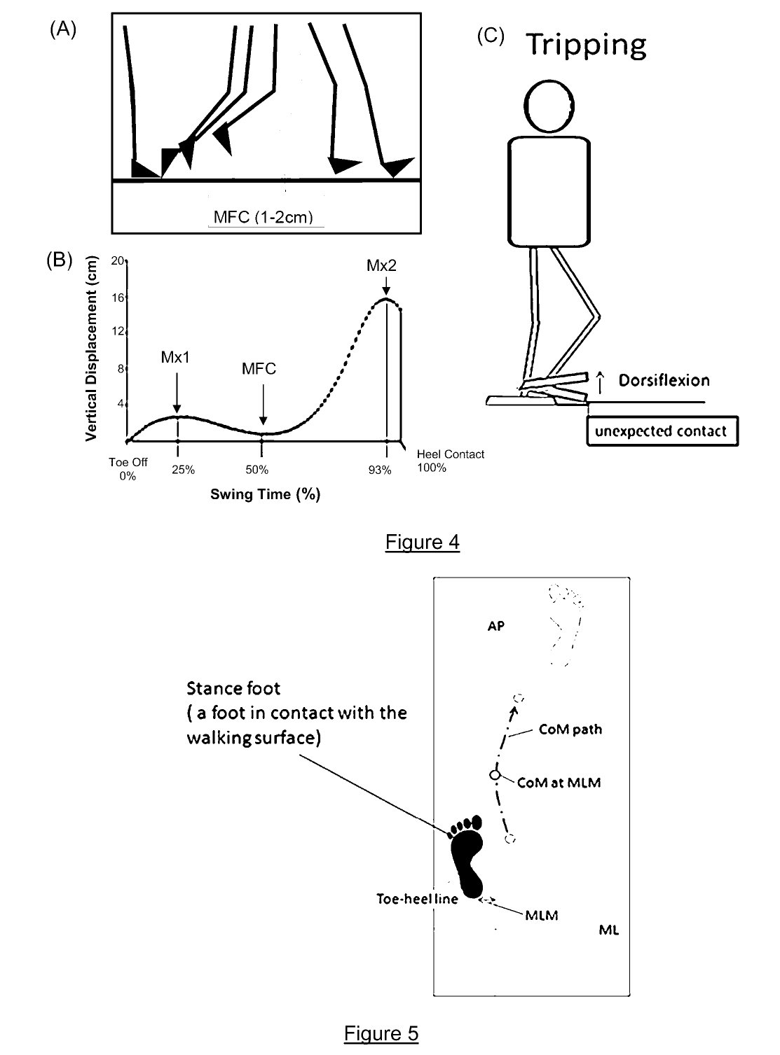

FIG. 4 provides three FIGS. 4A to 4C) which demonstrate how dorsiflexion support of the insole of the present invention reduces tripping risks at minimum foot clearance (MFC). FIG. 4(A) shows an illustration of the movement of the MFC: the local minimum of vertical swing foot displacement at mid-swing. As illustrated, tripping is defined as an unexpected contact of a swing foot with a walking surface or an obstacle on it with the sufficient force that destabilises a walker. Minimum foot clearance (MFC) is a crucial part of a walking cycle with very low vertical clearance (˜1 to 2 cm). To prevent tripping, higher swing foot clearance is critical, and ankle dorsiflexion has been identified as the most effective primary lower limb joint motion that increases swing foot clearance at MFC. As shown in FIG. 4(C), dorsiflexion support provided by an insole of the present invention can increase swing foot clearance.

### Improvement of Sideways Balance

The insole of the present invention can improve sideways balance of a user when walking.

As illustrated in FIG. 5, lateral balance loss can be measured by recording the CoM movement lateral to the foot in contact with the walking surface (i.e., support foot boundary). In this Figure, AP=anterior-posterior; ML=medio-lateral. MLM=minimum medio-lateral distance between the Centre of Mass (CoM) and stance foot toe-heel line during the walking cycle. Minimum lateral margin (MLM) is defined when the CoM reaches the lateral safety boundary, defined by the line between the stance foot's toe and heel (for example as discussed in Nagano, H., Begg, R., and Sparrow, W. A. 2013. Ageing effects on medio-lateral balance during walking with increased and decreased step width. In proceedings of the 35th International Conference of the IEEE Engineering in Medicine and Biology Society (EMBS), IEEE. Osaka, Japan, 3-7 Jul. 2013). Sideways balance loss is defined when MLM is less than 0.

Eversion support of the insole of the present invention can assist CoM redirection away from the lateral safety boundary toward the opposite foot thereby increasing MLM to minimise sideways balance loss.

### Centre of Pressure (COP) Control by Eversion Support

The insole of the present invention can improve center of pressure control of a user when walking.

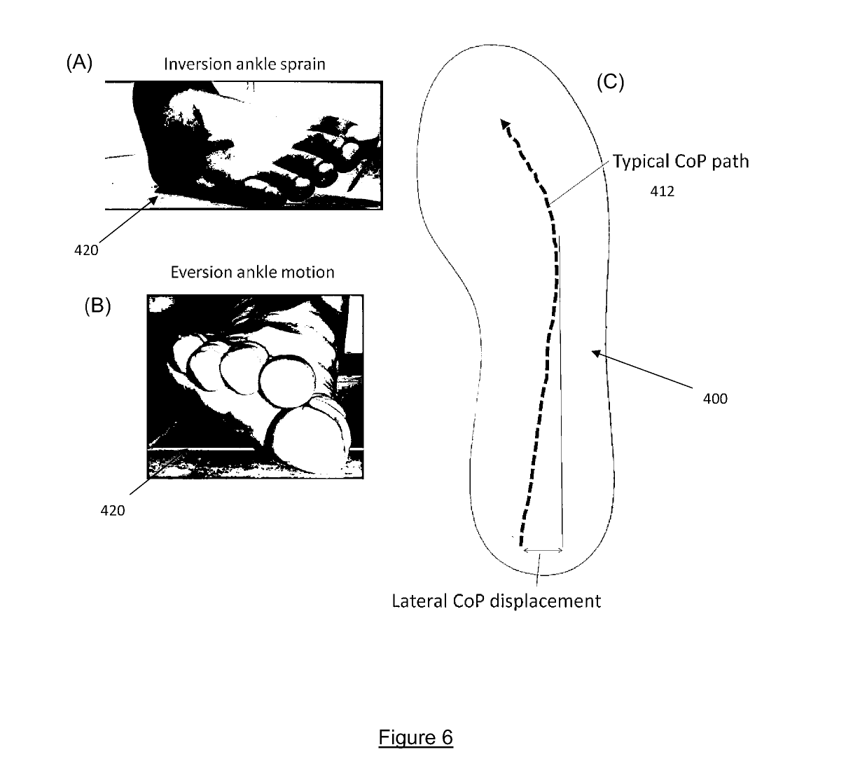

As illustrated in FIG. 6A an ankle inversion sprain 420 can lead to severe acute injuries such as ruptures of joint ligaments and it also can cause loss of sideways balance. This occurs when the foot's CoP displacement deviates excessively to the lateral direction. FIG. 6B shows related eversion ankle motion.

Eversion support of the insole of the present invention can help to regulate lateral CoP displacement, and control the CoP path 412 of a user's foot 400 as shown in FIG. 6C.

### Greater Mechanical Energy Efficiency During Loading Response

The insole of the present invention can improve mechanical energy efficiency, which may be useful for relieving conditions such as lower limb joint osteoarthritis.

Impact generated by foot strike can be partially oscillated to the opposite foot and can be utilised for the opposite foot's toe-off. The percentage of mechanical energy oscillation can be described by an established computational method to calculate recovery rate as indicated below. A more efficient loading response accompanies reduced impact transferred to lower limb joints.

Recovery rate (%)=100*[ΔKE+ΔPE−Δ(TEc)]/(ΔKE+ΔPE)

where ΔPE/ΔKE/ΔTEc indicate positive increase in mechanical energy of CoM during double support phase of gait cycle; PE is potential energy, KE is kinetic energy, TEc is the sum of KE and PE.

A dorsiflexed ankle at heel contact can stretch the Achilles tendon and absorb impact as elastic energy, which is later released toward opposite toe-off for maximum mechanical energy efficiency. More dorsiflexed heel contact, as provided by the insole of the present invention, also prolongs time to foot flat, which is biomechanically advantageous in dissipating peak force over longer period of time. Eversion support from the insole of the present invention can also facilitate the natural loading of a foot immediately following heel contact.

### Foot Pressure Distribution

The insole of the present invention can modify and/or improve foot pressure distribution of a user when walking.

Foot pressure distribution is possible by (semi) moulding of the insole surface to maximise the contact area with the foot (e.g., arch-support), as for example described above for the insole of the present invention. Dorsiflexion support provided by the insole of the present invention can also shift the concentration of foot pressure to the posterior direction, to move the pressure away from the common site of ulcer development.

### Other Effects of Dorsiflexion and Eversion on Walking Patterns

Ankle joint motions supported by footwear-insole intervention will not change walking patterns to the extent where other associated gait problems may arise. In other words, the insole of the present invention is to control fine-ankle joint movement only by a small amount (i.e., less than 1 cm increase in MFC) without making any dramatic changes to natural walking patterns. Addition of dorsiflexion up to 5° has been reported to cause no substantial effects on joint moments and fundamental step cycle spatio-temporal gait parameters.

### EXAMPLES

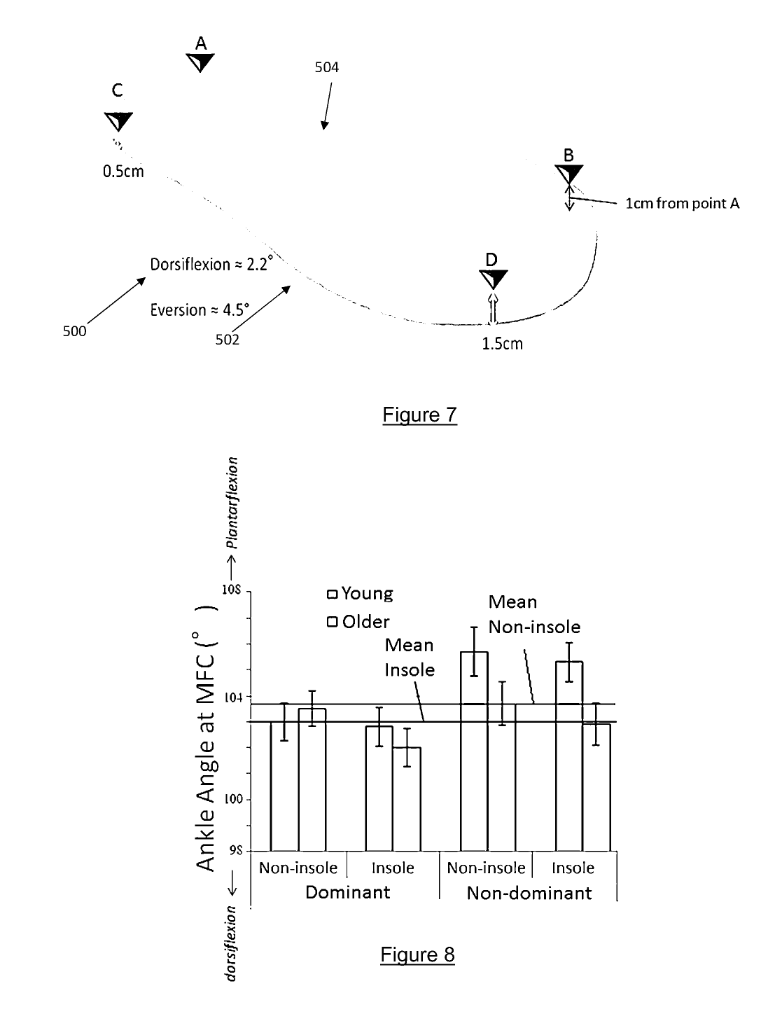

A combination of dorsiflexion and eversion has been applied to an experimental/test insole 500 (FIG. 7) to examine effects of combined joint motions on minimising tripping and lateral balance loss risks and enhanced stance foot loading and reduced impact transferred to the knees. The insole effects were also examined to confirm these ankle joint modifications would not cause fundamental changes to the gait patterns.

The experimental footwear-insole 500 is illustrated in FIG. 7. The experimental/tested footwear-insoles 500 were designed to support 2.2° dorsiflexion and 4.5° eversion at the static standing position provided by an incline between a planar base 502 and planar top surface 504 of the insole 500.

The exact dimensions of each experimental insole 500 varied in accordance with the size of the test subject's feet. The insole 500 was sized to fit the general shoe size of each test subject's feet. In an example of the 26 cm footwear-insole (FIG. 7), relative to the baseline height at the inner heel=0 cm (point A in FIG. 7), the most outer heel edge (point C in FIG. 7) was 0.5 cm higher and this eversion angle was applied across the entire insole surface. The most anterior inner surface, the inner toe (point B in FIG. 7) was elevated relative to the inner heel by 1.0 cm and the highest section was, accordingly, the most lateral and anterior toe section (point D in FIG. 7) at 1.5 cm higher to maintain the ankle joint angles.

A total of 30 young (18 to 35 yrs.) and 26 healthy older adults (>65 yrs.) participated in gait testing wearing both the flat shoe insole and the experimental insole 500, both the dominant and non-dominant limbs were tested separately. A total of 60 to 90 gait cycles were collected from both lower limbs of every participant per insole condition.

The results of the investigation is summarised in subsections below. All the noted differences were qualified as statistically significant.

### Tripping Risk Minimisation

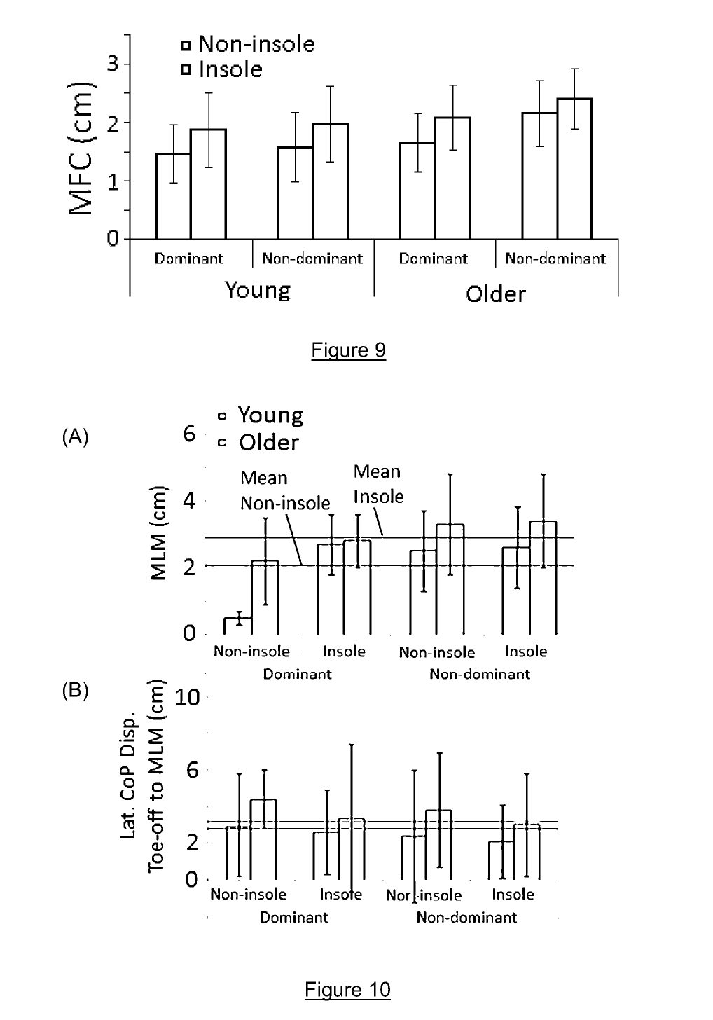

The tested insoles with 2.2° dorsiflexion and 4.5° eversion support at static standing increased dorsiflexion by 0.7° at MFC (FIG. 8) and elevated swing foot clearance by 0.36 cm (FIG. 9), a remarkable increase in swing foot clearance for tripping risk minimisation.

### Improving Sideways Balance

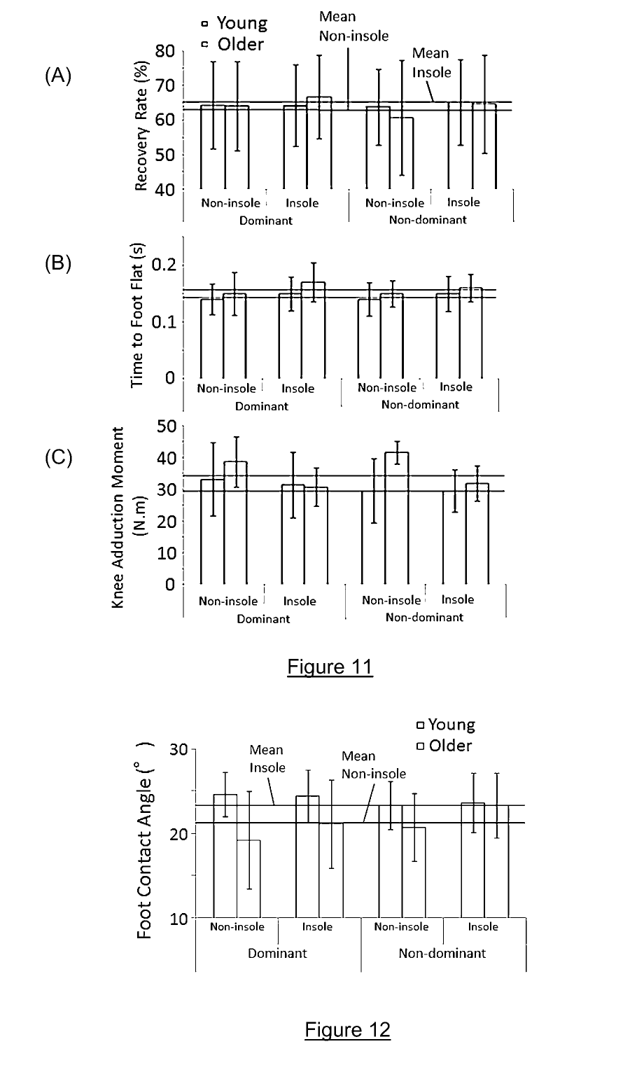

In combination with dorsiflexion, 4.5° eversion support regulated lateral movement of the CoM and reduced MLM by 0.75 cm (FIG. 10).

### Prevention of Inversion Sprain

CoP lateral displacement was reduced by 0.63 cm wearing the tested insoles, therefore lower risks of inversion sprain and associated lateral balance loss (FIG. 10).

### Energy Efficient Loading Response and Reduced Risks of Knee Osteoarthritis

A combination of dorsiflexion and eversion enhanced mechanical energy efficiency at heel contact, confirmed by 2% increase in recovery rate (FIG. 11). In addition, the insole increased foot contact angle especially for the older adults by greater than 2° (FIG. 12) and time to foot flat from heel contact was prolonged by 0.015 s (FIG. 11). A combination of these effects reduced impact transferred to lower limb joints, reflected in more than 20% reduction in knee adduction moment (FIG. 11).

### Gait Patterns not Affected by the Tested Insole

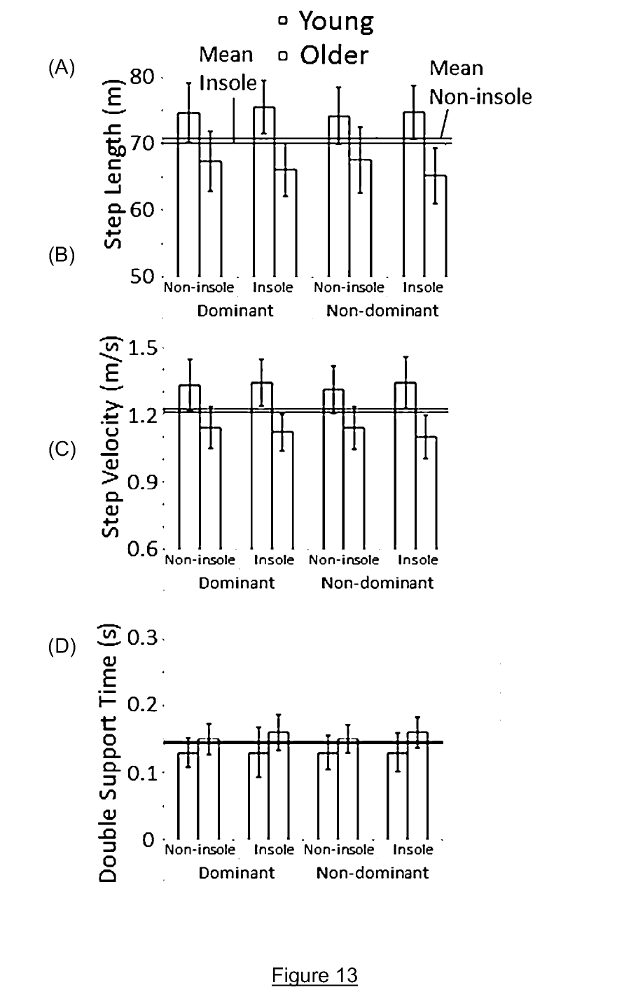

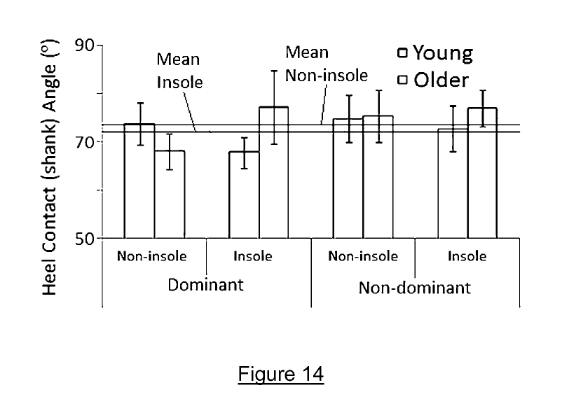

The tested insole did not change step velocity, length and double support time (FIG. 13). The tested insole did not have any effects on shank-floor contact angles (FIG. 14), an implication for not negatively increasing slipping risks.

It should be appreciated that the application of all the biomechanical modifications of the present invention on footwear-insoles is the standard recommendation to maximise the benefits. Inclination angles of the footwear-insole interface due to dorsiflexion and eversion can vary. The insole of the present invention may therefore include some but not all of the features discussed above. For example, the discussed moulding or semi-moulding and various sizes, number, diameters and materials of textures are optional features which can be included where considered useful or advantageous. For example, texture installation is to be avoided in the presence of, for example, foot ulcer. Arch-support is not applicable for a foot without an arch.

Those skilled in the art will appreciate that the invention described herein is susceptible to variations and modifications other than those specifically described. It is understood that the invention includes all such variations and modifications which fall within the spirit and scope of the present invention.

Where the terms “comprise”, “comprises”, “comprised” or “comprising” are used in this specification (including the claims) they are to be interpreted as specifying the presence of the stated features, integers, steps or components, but not precluding the presence of one or more other feature, integer, step, component or group thereof.

1. A footwear-insole for modifying a user's gait, wherein the footwear insole is configured to extend under a user's foot and provide greater than 0 to 5.0° dorsiflexion support and greater than 0 to 5.0° eversion support.

2. A footwear-insole according to claim 1, wherein the insole provides from 0.5° to 3.5° dorsiflexion support, preferably from 1.0° to 3.0° dorsiflexion support, more preferably from 2.0° to 2.5° dorsiflexion support, and yet more preferably about 2.2° dorsiflexion support.

3. A footwear-insole according to claim 1 or 2, wherein the insole provides from 2.0° to 5.0° eversion support, preferably from 3.0° to 5.0°, more preferably from 3.5° to 5.0° eversion support, yet more preferably from 4.0° to 5.0° eversion support, and yet more preferably about 4.5° eversion support.

4. A footwear-insole according to any preceding claim, wherein the insole is configured to be generally foot shaped.

5. A footwear-insole according to any preceding claim, wherein the insole is configured to seat under a user's foot.

6. A footwear-insole according to claim 5, wherein the insole is configured to be inserted into footwear.

7. A footwear-insole according to any preceding claim, wherein the insole extends from the heel to the outer toe of a user's foot, the insole providing a longitudinal and a lateral inclination from an inner heel section of the insole to an outer toe section of the insole to respectively provide dorsiflexion and eversion support.

8. A footwear-insole according to any preceding claim, wherein the insole includes a substantially planar base and a top surface, the longitudinal and the lateral inclination being formed between the base and the top surface of the insole.

9. A footwear-insole according to any preceding claim, wherein the longitudinal inclination and the eversion angle lateral inclination are substantially the same across the entire insole where surface contours are not a substantial factor.

10. A footwear-insole according to any preceding claim, wherein the insole includes the following reference points:
A=the most inner medial part of the insole within one fifth of the posterior part of the insole;
B=the most inner medial part of the insole within one-fifth of the anterior part of the insole;
C=the most outer lateral part of the insole within one-fifth of the posterior part of the insole; and
D=the most outer lateral part of the insole within one-fifth of the anterior part of the insole;
and wherein the dorsiflexion angle of A to B is approximately equal to the dorsiflexion angle of C to D; and the eversion angle of A to C is approximately equal to the eversion angle of B to D.

11. A footwear-insole according to any preceding claim, further comprising a pair of insoles, a left foot insole and a right foot insole, wherein the dorsiflexion support and eversion support are tailored for each foot of a user.

12. A footwear-insole according to any preceding claim, wherein the insole includes at least one layer of a viscoelastic material, preferably a closed cell foam, more preferably an ethylene vinyl acetate (EVA) foam.

13. A footwear-insole according to any preceding claim, wherein the dorsiflexion support angle is selected to assist with ankle joint dorsiflexion during locomotion.

14. A footwear-insole according to any preceding claim, wherein the eversion support angle is selected to provide improved sideways balance during locomotion.

15. A footwear-insole according to any preceding claim, wherein at least a portion of a top surface of the insole is moulded to fit a user's foot shape.

16. A footwear-insole according to claim 15, wherein the top surface of the insole includes arch support.

17. A footwear-insole according to any preceding claim, wherein the insole includes a top surface including at least one textured section.

18. A footwear-insole according to claim 17, wherein the textured section has a texture height of from 1 mm to 20 mm

19. A footwear-insole according to claim 17 or 18, wherein the textured section has a diameter from 5 mm to 30 mm,

20. A footwear-insole according to any one of claims 17 to 19, wherein the textured sections are configured on the top surface of the insole in at least one of the following configurations:
at least two, preferably multiple, textured sections aligned along the CoP path of the insole;
at least two parallel lines of multiple textured sections aligned along and with the CoP path of the insole;
at least two textured sections located in a Metatarsal region of the insole; or
an array of textured sections laterally spread along one lateral half of the insole.

21. A footwear-insole according to any preceding claim, wherein the configuration of the texture sections is different on the insoles of a user's two feet.

22. A method of controlling the gait of a user to reduce the risk of the user falling from or during locomotion comprising providing at least one footwear-insole according to any one of the preceding claims in the user's shoe.

23. A method of modifying the gait of a patient to reduce the risk of falling from or during locomotion, the method comprising:
measuring movement of a patient's minimum foot clearance (MFC) to determine a suitable dorsiflexion support angle to enhance ankle joint dorsiflexion and thereby increase swing foot clearance of the patient at MFC during locomotion;
measuring lateral movement of a patient's body centre of mass (CoM) during locomotion to determine an eversion support angle which redirects the CoM away from a lateral safety boundary toward the opposite foot; and
providing a footwear insole in a user's footwear configured to extend under a user's foot which provides the determined dorsiflexion support angle and eversion support angle, wherein the footwear insole provides greater than 0 to 5.0° dorsiflexion support and greater than 0 to 5.0° eversion support.

24. A method according to claim 23, wherein the footwear insole comprises an insole footwear-insole according to any one claims 2 to 21.
